(19) United States
(12) Patent Application Publication
(10) Pub. No.: US 2017/0000681 A1
(43) Pub. Date: Jan. 5, 2017

(54) LEG BRACE SUPPORT

(71) Applicant: Sam Adams, Scottsbluff, NE (US)

(72) Inventor: Sam Adams, Scottsbluff, NE (US)

(21) Appl. No.: 14/788,430

(22) Filed: Jun. 30, 2015

(51) Int. Cl. A61H 3/00; A61F 5/37

(52) U.S. Cl. CPC A61H 3/00; A61F 5/37; A61H 2003/007; A61H 2205/10

(57) ABSTRACT

Disclosed is a system and method for maintaining a leg brace or leg splint in a proper position respective to the leg of a patient.

### FIELD OF THE INVENTION

The field of the invention relates to leg braces and/or leg splints.

### BACKGROUND OF THE INVENTION

Leg braces and leg splints are devices used in the medical industry to aid a patient in maintaining their leg in a desired position or angle. For instance, after a patient has received medical attention to a portion of their leg, the attending physician or medical personnel may indicate that proper healing of the patient's leg will be quicker, or more permanent, if the patient does not bend their leg for a certain period of time. Accordingly, keeping a leg in a straight, unbended, position for a prescribed period of time may aid in the healing process.

Leg braces and splints may be constructed of a plastic shell, with cushioning pads, that immobilize, protect and support a patient's leg. The leg braces and splints may apply gently compression to speed recovery and may be removed to allow rehab exercises and for hygiene purposes. The leg braces and splints may be applied over a patient's clothing so that a patient may remain clothed during the removal process.

### SUMMARY OF THE INVENTION

An embodiment of the invention may therefore comprise a support for a leg cast, the support comprising a first support structure, comprising an upper support section, a lower support section and an engagement portion, a second support structure, comprising an upper support section, a lower support section and an engagement portion, a first hinge that connects the first support structure upper support section to the first support structure lower support section, a second hinge that connects the second support structure upper support section to the second support structure lower support section, and a platform connected to first support structure lower support section and to the second support structure lower support section.

An embodiment of the invention may further comprise a method of supporting a leg cast, the method comprising engaging a lower edge of the leg cast with an engagement portion of a first support structure, engaging the lower edge of the leg cast with an engagement portion of a second support structure, and providing a platform connected to the first support structure and the second support structure distally from the lower edge of the leg cast.

### BRIEF DESCRIPTION OF THE DRAWINGS

FIG. 1 shows a leg in a soft cast.

FIG. 2 shows a leg in a soft cast with a leg brace support.

FIG. 3 shows a leg brace support u-shaped engagement portion.

FIG. 4 shows a leg support brace with an alternate engagement section.

FIG. 5 shows a lower part of a leg brace support.

### DETAILED DESCRIPTION OF THE EMBODIMENTS

In an embodiment of the invention, a system and method is provided to maintain a leg brace or splint in a desired position in regard to a patient's leg. The leg brace or splint may sometimes be referred to as a soft cast. For purposes of this description, leg brace, leg splint, soft cast and leg immobilizer are synonymous. The leg brace or splint is generally applied to a patient's leg over the patient's clothing to minimize movement of the leg. Essentially, the leg brace or splint is intended to keep the patient from bending their leg. Generally, as a patient that is mobile and wearing a leg brace or splint moves around, the leg brace or splint may slide down the patient's leg. The patient, responsively, must continually pull the leg brace or splint back into a proper position. A patient may do this hundreds or thousands of times during any particular day. The sliding down of the leg brace or splint, and responsive pulling back up of the leg brace or splint, may cause irritation and chafing in the patient. Further, being required to continuously pull the soft cast back into position imposes unnecessary limits on a patient in terms of their mobility and ability to use their hands for other purposes.

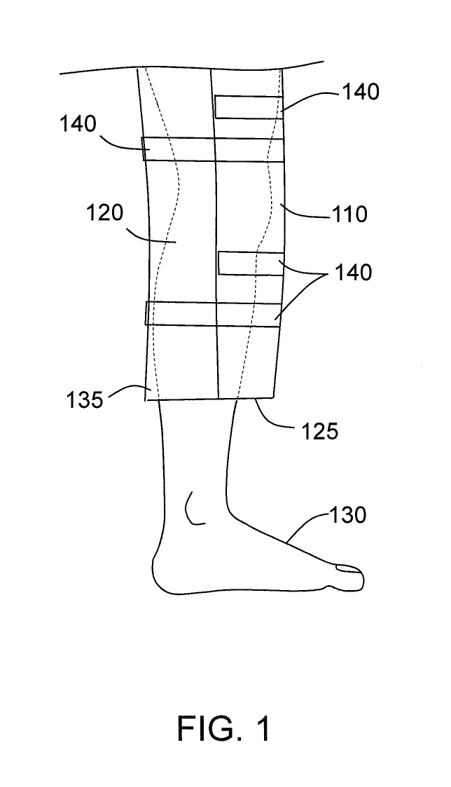

FIG. 1 shows a leg in a soft cast. The soft cast 110 is shown with a patient's leg 120 extending from a bottom portion 125 of the soft cast 110. The patient's foot 130 is below the bottom portion 125 of the soft cast 110 and not encompassed by the soft cast 110. A plurality of straps 140 are used with the soft cast 110 to close the soft cast 110 on the patient's leg 120. It is intended that the soft cast 110 shown in the FIG. 1 can be any soft cast. The soft cast may be comprised of a stiff material that disallows bending of the leg 120 at the knee. Further, it is understood that a suitable padding material 135 may be disposed on the stiff material so that the leg is cushioned. The padding material 135 may also be on the outside of the soft cast 110. Those skilled in the art will understand the construction of a soft casts. The embodiments in the invention are not intended to be limited to any particular style of soft cast. It is understood that other methods of closing the soft cast 110 may be used. These may include clips, Velcro straps, buckles or other method that those skilled in the art will understand.

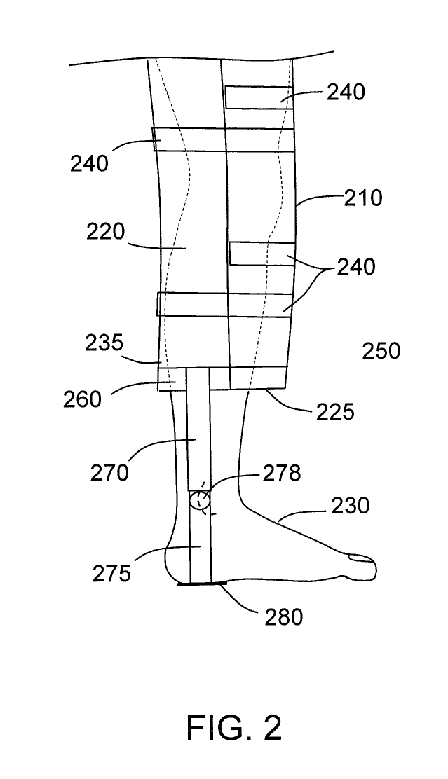

FIG. 2 shows a leg in a soft cast with a leg brace support. The soft cast 210 is shown with a patient's leg 220 extending from a bottom portion 225 of the soft cast 210. The patient's foot 230 is below the bottom portion 225 of the soft cast 210 and not encompassed by the soft cast 210. A plurality of straps 240 are used with the soft cast 210 to close the soft cast 210 on the patient's leg 220. In the embodiment of the invention shown in FIG. 2, an upper support section 270 engages in a hinged fashion with a lower support section 275. A hinge device 278 connects the upper support section 270 with the lower support section 275. The hinge 278 allows the lower support section 275 to rotate forward (toward the toes of the foot 230) and backward (toward the heel of the foot 230). The hinge 278 may be as simple as a screw that connects the upper section 270 with the lower section 275. Those skilled in the art will understand the manners in which a lower section and an upper section can be combined.

The forward and backward swing of the lower support section 275 may be intentionally restricted in order to aid in stabilization of the knee by the leg brace 210. The upper support portion 270 engages the leg brace 210 at the bottom 225. The engagement portion 250 of the upper portion 270 may comprise an extension of the upper portion 270 and have a contact (not shown) on the inside of the engagement portion 250. The contact may be a material that contacts the leg brace 210 and causes friction. The friction disallows the leg brace 210 to slide downward. The contact may be made of any suitable material. The contact may be made of a Velcro type material that adheres to the leg brace 210. A support strap 260 may be applied to the lower portion of the leg brace 210 in order to apply pressure to the contact to aid in maintaining friction between the contact and the leg brace 210.

The engagement portion 250 may be alternately formed. The engagement portion 250 may form a u-shaped portion so that the leg brace 210 slides down into the u-shape. In combination with the u-shape, there may be a friction mechanism, such as Velcro, to keep the leg support engagement portion 250 and the leg brace 210 from sliding with respect to each other.

Continuing with FIG. 2, a platform 280 connects to the lower support portion 275. The platform 280 allows for the foot 230 to sit on a flat surface and engage with the brace support. The platform 280 may be a flat surface or may be molded to approximate the bottom of the foot 230. The lower section 275 may connect to the platform 280 by any means. For instance, the platform 280 and the lower section 275 may be molded. The platform 280 and the lower section 275 may be connected otherwise to each other. Those skilled in the art will understand the manners by which a platform 280 and a lower section can be connected. The embodiments of the invention are not limited to any particular means of connection.

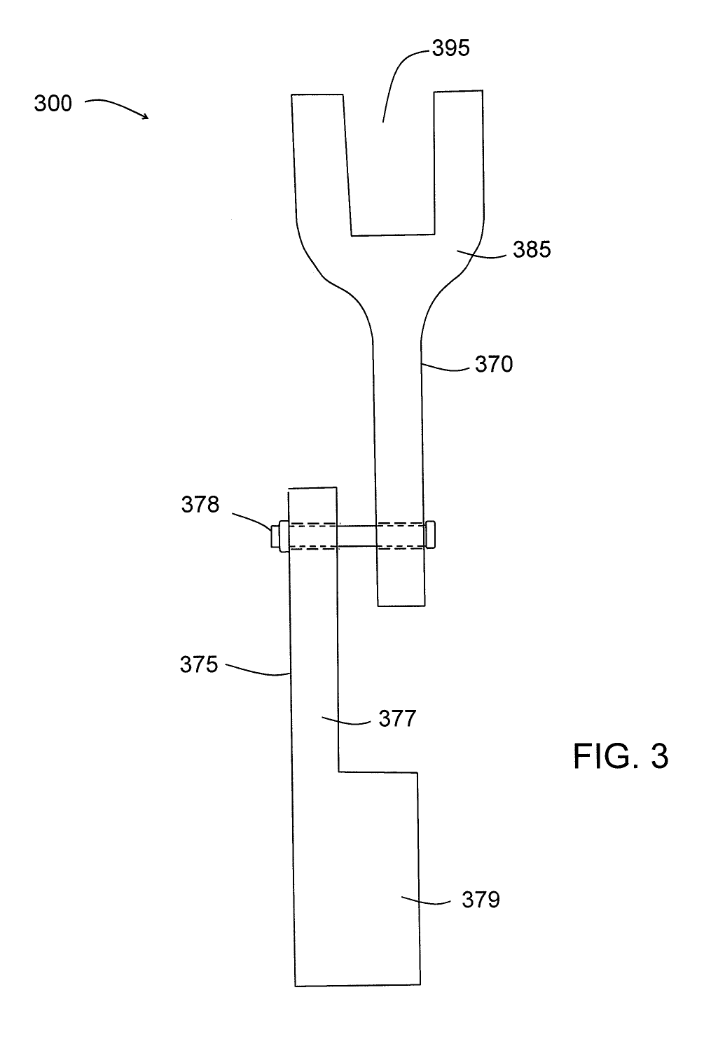

FIG. 3 shows a leg brace support u-shaped engagement portion. A leg brace support 300 has an upper support section 370 and a lower support section 375. The upper support section 370 is connectable to the lower support section 375 by a bolt 378. It is understood that other means of connection are possible. For instance, a rivet type connection may be used to connect the upper support section 370 to the lower support section 375. Those skilled in the art will understand the various mechanisms for connecting a first section to a second section. The lower support section 375 has a first ankle 377 clearance section and a second foot section 379. The ankle clearance section 377 allows the lower support section 375 to rotate in relation to the upper support section 375 without contacting a person's ankle. The foot section 379 is thicker than the ankle clearance section 377 to then allow the lower support section 377 to snugly fit against a person's foot. It is understood that the ankle clearance section 377 may be formed alternatively. The ankle clearance section 377 may be formed by curving the lower support section 375 outward from a person's ankle to allow movement of the lower support section 375 without contact with the person's ankle. A u-shaped engagement portion 395 is at a top end of the upper support section 370. The engagement portion 395 will naturally be distally located from the foot section 379 of the lower support section 375. The u-shaped engagement portion 395 comprises a gap that allows the lower edge (225 in FIG. 2) of a immobilizing cast 210 to drop into. The u-shaped engagement portion 395 disallows movement of the immobilizing cast 210. Accordingly, the immobilizing cast 210 will not be allowed to slide downward. As discussed herein, a strap (not shown in FIG. 3) may be used to wrap around the lower edge 225 of the immobilizing cast 210. The strap would likewise wrap the leg brace support 300 in order to maintain the support in a suitable position. Likewise, an adherent material may be placed on the inner portions of the u-shaped engagement portion to mildly adhere to the lower edge 225 of the immobilizing cast 210. The adherent material will create a friction between the immobilizing cast 210 and the leg brace support 300 to minimize movement of the leg brace support 300. The adherent material may be a Velcro type material. Those skilled in the art will recognize the types of materials suitable to create a friction or otherwise hold the leg brace support 300 relatively still in relation to the immobilization cast 210.

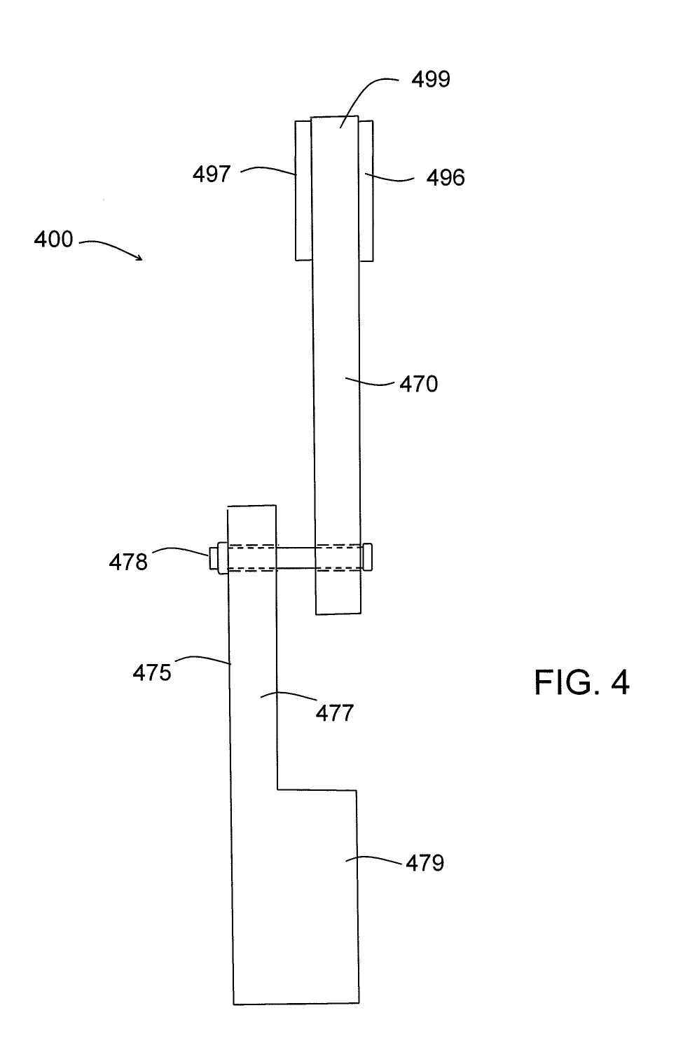

The leg brace support 300, 400 of FIG. 3 and FIG. 4 may be constructed of any material that provides physical support to an immobilizing cast 210. The leg brace support 300, 400 accordingly needs to be constructed of a material that is stiff enough to withstand the weight of an immobilizing cast 210. The leg brace support 300, 400 may be constructed of a plastic that provides adequate support. The leg brace support 300, 400 may be constructed of a metal that provides adequate support. Those skilled in the art will recognize the variety of materials and composites that are suitable for manufacture of a leg brace support 300, 400. The upper support portion 370, 470 may be constructed of a different material than the lower support portion 375, 475. The lower support portion 375, 475 may be constructed of a softer material that still provides adequate vertical support but which may be more suitable to nearness and contact with a person's ankle and foot.

FIG. 4 shows a leg support brace with an alternate engagement section. A leg brace support 400 has an upper support section 470 and a lower support section 475. The upper support section 470 is connectable to the lower support section 475 by a bolt 478. The lower support section 475 has a first ankle 477 clearance section and a second foot section 479. The ankle clearance section 477 allows the lower support section 475 to rotate in relation to the upper support section 475 without contacting a person's ankle. The foot section 479 is thicker than the ankle clearance section 477 to then allow the lower support section 477 to snugly fit against a person's foot. It is understood that the ankle clearance section 477 may be formed alternatively. The ankle clearance section 477 may be formed by curving the lower support section 475 outward from a person's ankle to allow movement of the lower support section 475 without contact with the person's ankle. An engagement portion 499 has an adherent material 496 on an inside face. The engagement portion 499 may also have an adherent material 497 on an outside face. For this description, the terms inside and outside may simply mean inside facing to a person or outside facing away from a person. The adherent material 496, 497 will create a friction between the immobilizing cast 210 and the leg brace support 400 to minimize movement of the leg brace support 400 and to hold the immobilizing cast 210 up. The adherent material may be a Velcro type material. Those skilled in the art will recognize the types of materials suitable to create a friction or otherwise hold the leg brace support 400 relatively still in relation to the immobilization cast 210. As discussed herein, a strap (not shown in FIG. 4) may be used to wrap around the lower edge 225 of the immobilizing cast 210. The strap would likewise wrap the leg brace support 400 in order to maintain the support in a suitable position. The adherent materials 496, 497 may be on both the inner portion and the outer portion of the upper support portion 470. The adherent material 496, 497 may also be only the inner portion or the outer portion of the upper support portion 470. The adherent material may also be absent from the upper support portion 470. In such a case, the upper engagement portion 499 may fit into a pocket or similar construction in an immobilizing cast.

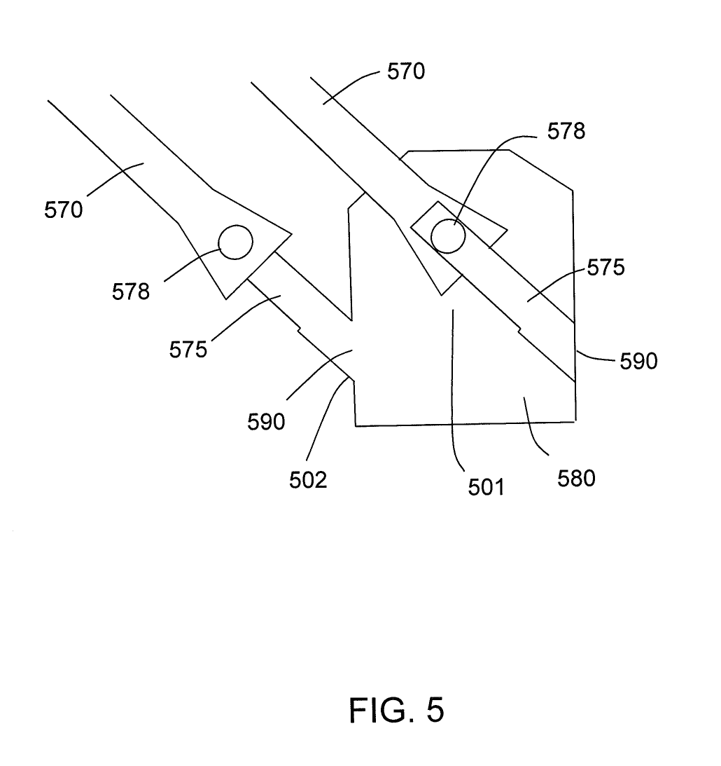

FIG. 5 shows a lower part of a leg brace support. The leg brace support 500 has a first side 501 and a second side 502. It is understood that the leg brace supports of FIGS. 1-4 likewise will have a first and second side. The first and second sides are essentially left and right sides, or are and inside and an outside with respect to inside the ankle and outside the ankle. The first side 501 and second side 502 have a lower support section 575, an upper support section 570, a hinge 578. The upper support section 570 may flare outward at and around the location of the hinge. Flaring the upper support section 570 at the hinge location may provide additional support. The lower support sections 575 connect to a platform 580. As discussed, the platform 580 provides a surface for a person's foot to engage. The platform 580 may be sized to fit inside a person's shoe. The platform 580 and lower support sections 575 may be sized to fit outside a person's shoe.

The foregoing description of the invention has been presented for purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

What is claimed is:

1. A support for a leg cast, said support comprising:
a first support structure, comprising an upper support section, a lower support section and an engagement portion;
a second support structure, comprising an upper support section, a lower support section and an engagement portion;
a first hinge that connects said first support structure upper support section to said first support structure lower support section;
a second hinge that connects said second support structure upper support section to said second support structure lower support section; and
a platform connected to first support structure lower support section and to said second support structure lower support section.

2. The support of claim 1, wherein said first support structure engagement portion comprises a u-shape enabled to engage a lower edge of said leg cast and said second support structure engagement portion comprises a u-shape to engage a lower edge of said leg cast.

3. The support of claim 1, wherein said first support structure engagement portion comprises at least one adherent material on one of an inside face and an outside face and said second support structure engagement portion comprises at least one adherent material on one of an inside face and an outside face.

4. The support of claim 3, wherein said adherent material is Velcro.

5. The support of claim 1, said support further comprising a strap wrap-able around said leg cast.

6. The support of claim 1, wherein at least one of said first support structure lower support section and said second support structure lower support section comprises an ankle clearance area.

7. A method of supporting a leg cast, said method comprising:
engaging a lower edge of said leg cast with an engagement portion of a first support structure;
engaging said lower edge of said leg cast with an engagement portion of a second support structure; and
providing a platform connected to said first support structure and said second support structure distally from said lower edge of said leg cast.

8. The method of claim 7, wherein said first support structure comprises an upper portion and a lower portion and said second support structure comprises an upper portion and a lower portion, said method further comprising:
hinging said first support structure upper portion to said first support structure lower portion; and
hinging said second support structure upper portion to said second support structure lower portion.

9. The method of claim 7, wherein said engagement portion of said first support structure comprises a u-shape and said engagement portion of said second support structure comprises a u-shape.

10. The method of claim 7, wherein said engagement portion of said first support structure comprises at least one adherent material on one of an inside face and an outside face and said engagement portion of said second support structure comprises at least one adherent material on one of an inside face and an outside face.

11. The method of claim 10, wherein said adherent material is Velcro.

12. The method of claim 7, said method further comprising wrapping a strap around said lower edge of said leg cast.
